SEQUENCE LISTING (15 sequences)

SEQ ID NO: 1 - DNA, 20 bp, Artificial Sequence
  Description of Artificial SequencePCR primer for Salmonella sp.
acagcaaaatgcggatgctt

SEQ ID NO: 2 - DNA, 20 bp, Artificial Sequence
  Description of Artificial SequencePCR primer for Salmonella sp.
gcgcgctcagtgtaggactc

SEQ ID NO: 3 - DNA, 34 bp, Artificial Sequence
  Description of Artificial SequenceDetection probe for Salmonella sp.
gcaatccgttagcgctaaagatattctgaatagt

SEQ ID NO: 4 - DNA, 33 bp, Artificial Sequence
  Description of Artificial SequenceDetection probe for Salmonella sp.
ttggtattagcagcagtaaagtcagtgacctgg

SEQ ID NO: 5 - DNA, 18 bp, Artificial Sequence
  Description of Artificial SequencePCR primer for E. coli O157H7
tggtacgggtaatgaaaa

SEQ ID NO: 6 - DNA, 19 bp, Artificial Sequence
  Description of Artificial SequencePCR primer for E. coli O157H7
aatagcctggtagtcttgt

SEQ ID NO: 7 - DNA, 19 bp, Artificial Sequence
  Description of Artificial SequenceDetection probe for E. coli O157H7
cgcagtcagggcggtcaga

SEQ ID NO: 8 - DNA, 20 bp, Artificial Sequence
  Description of Artificial SequenceDetection probe for E. coli O157H7
tcagcatagcggaagccaaa

SEQ ID NO: 9 - DNA, 20 bp, Artificial Sequence
  Description of Artificial SequencePCR primer for Listeria monocytogenes
atttagtggaaccgtgacgc

SEQ ID NO: 10 - DNA, 19 bp, Artificial Sequence
  Description of Artificial SequencePCR primer for Listeria monocytogenes
gatgtcatttgtcggcatt

SEQ ID NO: 11 - DNA, 22 bp, Artificial Sequence
  Description of Artificial SequenceDetection probe for Listeria monocytogenes
agctaagcccgtaaaagaaggt

SEQ ID NO: 12 - DNA, 26 bp, Artificial Sequence
  Description of Artificial SequenceDetection probe for Listeria monocytogenes
acacatttgttggttggtttgatgcc

SEQ ID NO: 13 - DNA, 251 bp, Salmonella sp.
  misc_feature (1)..(251): Fragment of sipB-sipC region
acagcaaaatgcggatgcttcgcgttttattctgcgccagagtcgcgcataaaaactgccaaaataaagggagaaaaatatgttaattagtaatgtgggaataaatcccgccgcttatttaaataatcattctgttgagaatagttcacagacagcttcgcaatccgttagcgctaaagatattctgaatagtattggtattagcagcagtaaagtcagtgacctggggttgagtcctacactgagcgcgc

SEQ ID NO: 14 - DNA, 361 bp, E. coli O157H7
  misc_feature (1)..(361): Fragment of eae gene
tggtacgggtaatgaaaatgatctcctttactcaatgcagttccgttatcagtttgataaatcgtggtctcagcaaattgaaccacagtatgttaacgagttaagaacattatcaggcagccgttacgatctggttcagcgtaataacaatattattctggagtacaagaagcaggatattctttctctgaatattccgcatgatattaatggtactgaacacagtacgcagaagattcagttgatcgttaagagcaaatacggtctggatcgtatcgtctgggatgatagtgcattacgcagtcagggcggtcagattcagcatagcggaagccaaagcgcacaagactaccaggctatt

SEQ ID NO: 15 - DNA, 217 bp, Listeria monocytogenes
  misc_feature (1)..(217): Fragment of internalin operon
atttagtggaaccgtgacgcagccacttaaggcaatttttaatgttaagtttcatgtggacggcaaagaaacaaccaaagaagtggaagctgggaatttattgactgaaccagctaagcccgtaaaagaaggtcacacatttgttggttggtttgatgcccaaacaggcggaactaaatggaatttcagtacggataaaatgccgacaaatgacatc

(19) United States
(12) Patent Application Publication
(10) Pub. No.: US 2006/0240442 A1
(43) Pub. Date: Oct. 26, 2006

(54) Methods and oligonucleotides for the detection of Salmonella SP., E coli 0157:H7, and Listeria monocytogenes

(76) Inventor: Dirk N. Vevea, Hewitt, WI (US)

Correspondence Address: RYAN KROMHOLZ & MANION, S.C., POST OFFICE BOX 26618, MILWAUKEE, WI, 53226, US

(21) Appl. No.: 11/110,510

(22) Filed: Apr. 20, 2005

(51) Int. Cl. C12Q 1/68; C12P 19/34

(52) U.S. Cl. 435/6; 435/91.2

(57) ABSTRACT

A method for detecting a Salmonella species, E. coli 0157:H7, or Listeria monocytogenes is disclosed. The method involves amplifying a genomic nucleotide sequence of a corresponding species and detecting the amplification product. Various primers and probes that can be used in the method are also disclosed. In one embodiment, the amplification step of the method is accomplished by real-time PCR and the amplification product is detected by fluorescence resonance energy transfer using a pair of labeled polynucleotides.

### RELATED APPLICATIONS

This application claims the benefit of U.S. application Ser. No. 10/178,331 filed 21 Jun. 2002, which claims the benefit of U.S. application Ser. No. 60/300,199, filed 22 Jun. 2001.

### BACKGROUND OF THE INVENTION

Federal and state health and safety standards mandate that industrial food service companies and manufacturing facilities perform routine testing for common bacteria, such as Salmonella species, E. coli 0157:H7, and Listeria monocytogenes, that cause food-borne illnesses. As a safety precaution, companies are required to perform testing on each batch or lot of food prior to the food reaching the public. Several methods are currently available for industrial testing of bacteria in the food service industry.

However, there are currently severe limitations on the tests available to the industry. Present methods utilized as industry standards require 2-5 days to perform. For example, the most widely used methods for detection of Salmonella employ a pre-enrichment (day 1), a selective enrichment (day 2), and a final enrichment followed by an immunoassay requiring 10⁵ organisms (day 3); the most widely used methods of detection of E. coli 0157:H7 employ a selective enrichment (8-28 hours) and an immunoassay requiring 10⁵ organisms; the most widely used methods of detection of Listeria monocytogenes employ a pre-enrichment (26-30 hours), an enrichment (22-26 hours), and an immunoassay requiring 10⁵ organisms. For the detection of E. coli 0157:H7 and Listeria monocytogenes, all samples that are suspected as positive by the immunoassay must be confirmed by culture methods (1-3 days for E. coli 0157:H7 and 4-5 days for Listeria monocytogenes). Thus, in many cases, the food suppliers must wait days for test results before shipping their already manufactured products. As a result, the company may lose profits from a reduced shelf life and the wait also increases the potential for food spoilage.

In addition, using methods now available in the art, the organism needs to be cultured to a concentration of at least 10⁵/ml to be detected. Because the margin of error in detectability of the bacteria is high, false negative tests may result and a food poisoning outbreak may occur. The company is then forced to recall product that has already reached the consumer. This places the public at a great health risk. The manufacturer or producer is also forced to bear the costs of recall, and is at a risk for lawsuit or government mandated shutdown of production facilities.

Thus, there is a need for an inexpensive testing technology that provides a rapid turn-around time, and a high degree of accuracy and reproducibility, which will result in safer food manufacturing and preparation. Additionally, there is a need for a method that keeps pace with new manufacturing processes. Polymerase chain reaction (“PCR”) testing technology for food-borne pathogenic bacteria facilitates rapid and accurate testing for the manufacturers.

### SUMMARY OF THE INVENTION

The present invention provides a method for detecting a Salmonella species, E. coli 0157:H7, or Listeria monocytogenes. The method involves amplifying a genomic nucleotide sequence of a corresponding species and detecting the amplification product. The present invention also encompasses primers and probes that can be used in the method. The primers and probes can be provided in a detection kit.

In one embodiment, the amplification step of the method of the present invention is accomplished by real-time PCR and the amplification product is detected by fluorescence resonance energy transfer using a pair of labeled oligonucleotides.

It is a feature of the present invention that the genomic region from which a nucleotide sequence is amplified is involved in bacterial virulence.

It is an advantage of the present invention that the method of bacteria detection is sensitive.

It is another advantage of the present invention that the method of bacteria detection is fast.

Other objects, advantages, and features of the present invention will become apparent from the following detailed description of the invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

The present invention relates to the detection of bacterial pathogens in food or other materials with much greater sensitivity and speed than was heretofore possible. Primers have been identified which permit a rapid and sensitive type of polymerase chain reaction (PCR) to amplify target DNA if, and only if, one of the target pathogens is present in a sample. Probes are also identified which will bind to the amplified DNA products produced again if, and only if, the organism is present. The method has been implemented for Salmonella, E. coli 0157:H7, and Listeria monocytogenes.

As used herein, an “isolated nucleic acid” is a nucleic acid which may or may not be identical to that of a naturally occurring nucleic acid but which is isolated from a living host organism. When “isolated nucleic acid” is used to describe a primer or a probe, the nucleic acid is not identical to the structure of a naturally occurring nucleic acid spanning at least the length of a gene.

In one aspect, the present invention relates to nucleic acids that can be used as primers to amplify a genomic fragment isolated from Salmonella species, E. coli 0157:H7 or Listeria monocytogenes to detect the corresponding species Such a nucleic acid has a nucleotide sequence containing at least 12 consecutive nucleotides of SEQ ID NO:1 (5′ primer for Salmonella species), SEQ ID NO:2 (3′ primer for Salmonella species), SEQ ID NO:5 (5′ primer for E. coli 0157:H7), SEQ ID NO:6 (3′ primer for E. coli 0157:H17), SEQ ID NO:9 (5′ primer for Listeria monocytogenes), or SEQ ID NO:10 (3′ primer for Listeria monocytogenes). Preferably, the nucleic acid has a sequence that contains at least 15 or 18 consecutive nucleotides, and most preferably the full length, of the above-identified sequences.

In another aspect, the present invention relates to labeled nucleic acids that can act as probes to facilitate the detection of an amplification product of a Salmonella species, E. coli 0157:H7 or Listeria monocytogenes, obtained using the primers described above. The labeled nucleic acid probes work in pairs. One probe in each pair is labeled at the 3′ end and the other probe is labeled at the 5′ end. Each probe pair hybridize to the same strand of the amplification product. When hybridized to the amplification product, the 3′ end nucleotide of the 3′ end labeled nucleic acid probe and the 5′ end nucleotide of the 5′ end labeled nucleic acid probe are less than six nucleotides apart so that energy transfer occurs between the two labels resulting in an emission intensity change of at least one of the labels. The emission intensity change indicates the presence of the amplification product.

The labeled nucleic acid probes in each pair have nucleotide sequences containing at least 12 consecutive nucleotides of SEQ ID NO: 13 (for Salmonella species), the complement of SEQ ID NO:13 (for Salmonella species), SEQ ID NO:14 (for E. coli 0157:H7), the complement of SEQ ID NO:14 (for E. coli 0157:H7), SEQ ID NO:15 (for =9 Listeria monocytogenes), or the complement of SEQ ID NO: 15 (for Listeria monocytogenes). Preferably, the labeled nucleic acids in each probe pair have nucleotide sequences containing at least 15 or 18 nucleotides of the above-identified sequences. Most preferably, the labeled nucleic acids in each pair have the following pair of nucleotide sequences: SEQ ID NO:3 and SEQ ID NO:4 (for Salmonella species), the complement of SEQ ID NO:3 and the complement of SEQ ID NO:4 (for Salmonella species), SEQ ID NO:7 and SEQ ID NO:8 (for E. coli 0157:H7), the complement of SEQ ID NO:7 and the complement of SEQ ID NO: 8 (for E. coli 0157:H7), SEQ ID NO:11 and SEQ ID NO:12 (for Listeria monocytogenes), and the complement of SEQ ID NO:11 and the complement of SEQ ID NO:12 (for Listeria monocytogenes).

Any pair of labeling molecules that can undergo energy transfer when located close to each other (less than 6 nucleotides apart on a nucleotide sequence) to cause a change in emission intensity in at least one of the labeling molecules can be used to make the labeled nucleic acids described above. An example of a labeling molecule for one nucleic acid in a pair includes, but are not limited to, fluorescein. Examples of labeling molecules for the other nucleic acid in the pair include but are not limited to LC RED 640 (Roche Lightcycler), LC RED 705 (Roche Lightcycler).

In another aspect, the present invention relates to a kit for detecting at least one of a Salmonella species, E. coli 0157:H7 and Listeria monocytogenes. The kit contains a pair of nucleic acid primers and a pair of labeled nucleic acids, as described above, for one, two or all three of the above species. Other reagents for the amplification of a target DNA and the detection of the amplification product can also be included in the kit. The kit may also include positive and negative controls for the above species. The positive control can be any sample that contains a target DNA to be amplified, including the bacteria themselves, at an amount over the detection limit. The negative control is a sample that does not contain the target DNA to he amplified.

In another aspect, the present invention relates to an isolated nucleic acid the amplification of which allows detection of a Salmonella species, E. coli 0157:H7 or Listeria monocytogenes. Examples of such nucleic acids include those that contain SEQ ID NO:13, SEQ ID NO:14 or SEQ ID NO:15.

In still another aspect, the present invention relates to a method for detecting a Salmonella species, E. coli 0157:H7, or Listeria monocytogenes. The method involves amplifying a fragment of the genomic DNA specific to the above species and detecting the amplification product. Unique sequences that can be used to identify a Salmonella species, E coli 0157:H7 and Listeria monocytogenes include nucleotide 2314 to nucleotide 2547 (nucleotide 9 to nucleotide 243 of SEQ ID NO13) of the sipB-sipC region of the Salmonella genome (GenBank Accession No U25631), nucleotide 1185 to nucleotide 1532 (nucleotide 7 to nucleotide 354 of SEQ ID NO:14) of the eae gene of E. coli 0157:H7 (GenBank Accession No AF081182), and nucleotide 2995 to nucleotide 3196 (nucleotide 9 to nucleotide 210 of SEQ ID NO:15) of the internalin operon of Listeria monocytogenes (GenBank Accession No. AJ012346). Any genomic fragments that contain the above sequences can be amplified for detecting the above species. Given what is disclosed herein, a skilled artisan knows how to amplify a fragment that contains one of the above specific sequences and then detect the presence of an amplification product that contains the sequence. Examples of the primers that can he used in the method of present invention are described above.

The genomic sequences amplified and detected with the method of the present invention are from genomic regions that are involved in bacterial virulence. The sip proteins of the Salmonella species and the internalin proteins of Listeria monocytogenes are required for cell invasion; the EAE proteins of E. coli 0157:H7 are required for cell effacement and attachment. Thus, the method of the present invention detects bacteria that harbor virulent traits. Nonpathogenic strains of these species are not meant to be detected using this technique.

It is understood that the species specific sequences actually amplified in performing the method of the present invention may vary somewhat from the sequences described above. The variations may be caused by sequencing errors, strain-specific variations or some other reasons. The method of the present invention intends to encompass these variations.

In a specific embodiment, a fragment of genomic DNA specific to a species is amplified by real-time PCR and the amplification product is detected by fluorescence resonance energy transfer (FRET) using labeled nucleic acids described above as internal hybridization probes. In this embodiment, internal hybridization probes are included in the PCR reaction mixture so that product detection occurs as the product is formed, further reducing post-PCR processing time. Roche Lightcycler PCR instrument (U.S. Pat. No. 6,174,670) or other real-time PCR instruments can be used in this embodiment of the invention. PCR amplification of DNA allows for the increase in sensitivity to less than 10¹ organisms in comparison to 10⁵ organisms in standard immuno-detection methods presently used. Real-time PCR amplification and detection can reduce total assay time so that test results can be obtained within 12 hours.

The invention will be more fully understood upon consideration of the following non-limiting examples.

### EXAMPLE 1

### Detection of Salmonella Species

A sample of the food product was weighed out and mixed with Buffered Peptone Water. The ratio of the food product to Buffered Peptone Water was 25 to 225 (grams to mls). The mixture was then mechanically homogenized and incubated at 35+/−2° C. After six hours of incubation, 15 ml of mixture was removed and centrifuged at 2,500×g for 10 minutes. The supernatant was discarded and the pellet was resuspended in 200 ml of TE. The DNA was then extracted from the bacteria using either the Qiagen QIAamp DNA mini kit (Qiagen Inc., Valencia, Calif.) or Biotecon foodproof® extraction kit (Potsdam, Germany).

Next, PCR amplification and detection of amplification product were performed. The following oligonucleotides were designed to provide for the PCR amplification of a 250 bp product spanning from base 2305 to base 2555 of the sipB-sipC region of the Salmonella genome (GenBank Accession #U25631): forward 5′-ACAGCAAAATGCGGATGCTT-3′ (SEQ ID NO:1) and reverse 5′-GCGCGCTCAGTGTAGGACTC-3′ (SEQ ID NO:2).

In addition, internal hybridization probes were designed to allow for detection of the PCR product by fluorescence resonance energy transfer within the Roche Lightcycler. The sequence and modifications of the probes were: upstream 5′-(GCAATCCGTTAGCGCTAAAGATATTCTGAATAGT-Fluorescein-3′ (SEQ ID NO:3) and downstream 5′-LC RED64OTTGGTATTAGCAGCAGTAAAGTCAGTGACCTGG-Phos-3′ (SEQ ID NO:4). These probes were designed to anneal to the upper strand from positions 2464-2497 (upstream) and 2499-2531 (downstream). PCR optimization was then carried out to allow for rapid real-time amplification and detection in the Roche Lightcycler PCR instrument (U.S. Pat. No. 6,174,670). PCR amplification of DNA led to an increase in sensitivity to less than 10¹ organisms in comparison to 10⁵ organisms in standard prior art immuno detection methods. These hybridization probes provided a high degree of specificity and accurate detection of Salmonella isolates. No false positives were observed.

This test methodology detected Salmonella at the low pre-enrichment concentration range of 10⁰ organisms/mL-10¹ organisms/mL by amplification of DNA using oligonucleotides. Utilizing the Roche Lightcycler, which completed cycles in about 30 minutes, instead of hours or overnight, as in older thermocyclers, allowed test results to be obtained within 12 hours.

### EXAMPLE 2

### Detection of E. coli 0157:H7

A sample of the food product was weighed out and mixed with modified Trypticase Soy Broth. The ratio of the food product to modified Trypticase Soy Broth was 25 to 225 (grams to mLs). The mixture was then mechanically homogenized and incubated at 35+/−2° C. After six hours of incubation, 15 ml of mixture was removed and centrifuged at 2,500×g for 10 minutes. The supernatant was discarded and the pellet was re-suspended in 200 ml of TE. The DNA was then extracted from the re-suspended bacteria using either the Qiagen QIAamp DNA mini kit (Qiagen Inc., Valencia, Calif.) or Biotecon foodproof® extraction kit (Potsdam, Germany).

Next PCR amplification and detection of PCR amplification product were performed. The following oligonucleotides were designed to provide for the PCR amplification of a 361 bp product spanning from base 1179 to base 1539 of the eae gene of the E. coli 0157:H7 genome (GenBank Accession #AF081182): forward 5′-TGGTACGGGTAATGAAAA-3′ (SEQ ID NO:5) and reverse 5′-AATAGCCTGGTAGTCTTGT-3′ (SEQ ID NO:6).

In addition, internal hybridization probes were designed for detection of the PCR product by fluorescence resonance energy transfer within the Roche Lightcycler. The sequence and modifications of the probes were: upstream 5′-CGCAGTCAGGGCGGTCAGA-Fluorescein-3′ (SEQ ID NO:7) and downstream 5′-LC RED640TCAGCATAGCGGAAGCCAAA-Phos-3′ (SEQ ID NO:8). These probes were designed to anneal to the upper strand from positions 1477-1495 (upstream) and 1497-1516 (downstream). PCR optimization was then carried out to allow for rapid real-time amplification and detection in the Roche Lightcycler PCR instrument (U.S. Pat. No. 6,174,670) or other real-time PCR instrument. PCR amplification of DNA led to an increase in sensitivity to less than 10¹ organisms in comparison to 10⁵ organisms in standard prior art immuno detection methods. These hybridization probes provided a high degree of specificity and accurate detection of E. coli 0157:H7 isolates. No false positives were observed.

Utilizing the Roche Lightcycler, which completed cycles in about 30 minutes, instead of hours or overnight, as in older thermocyclers, allowed test results to he obtained within 12 hours.

### EXAMPLE 3

### Detection of Listeria monocytogenes

Two hundred and twenty five ml of Fraser broth was added to a sample of 25 grams of the food product. The mixture was then stomached and incubated at 30° C. After eight hours of incubation, 15 ml of mixture was removed and centrifuged at 2,500×g for 10 minutes. The supernatant was discarded and the pellet was resuspended in 200 ml TE. The DNA was then extracted from the resuspended bacteria using either the Qiagen QIAamp DNA mini kit (Qiagen Inc., Valencia, Calif.) or Biotecon foodproof® extraction kit (Potsdam, Germany).

Next, PCR amplification and detection of PCR amplification product were performed. The following oligonucleotides were designed to provide for the PCR amplification of a 217 bp product spanning from base 2987 to base 3203 of the internalin operon of the Listeria monocytogenes genome: forward 5′-ATTTAGTGGAACCGTGACGC-3′ (SEQ ID NO:9) and reverse 5′-GATGTCATTTGTCGGCATT-3′ (SEQ ID NO:10).

In addition, internal hybridization probes were designed to allow for detection of the PCR product by fluorescence resonance energy transfer within the Roche Lightcycler. The sequence and modifications of the probes were upstream 5′-AGCTAAGCCCGTAAAAGAAGGT-Fluorescein-3′ (SEQ ID NO:11) and downstream 5′-LC RED640-ACACATTTGTTGGflGGTTTGATGCC-Phos-3′(SEQ ID NO:12). These probes were designed to anneal to the upper strand from positions 3098-3119 (upstream) and 3121-3146 (downstream). PCR optimization was then carried out to allow for rapid real-time amplification and detection in the Roche Lightcycler PCR instrument (U.S. Pat. No. 6,174,670) or other real-time PCR instrument. These hybridization probes provided a high degree of specificity and accurate detection of Listeria monocytogenes isolates. No false positives were observed.

Utilizing the Roche Lightcycler, which completed cycles in about 30 minutes, instead of hours or overnight, as in older thermocyclers, allowed test results to be obtained within 12 hours.

### EXAMPLE 4

### Detection of Salmonella species

The present example further determines accuracy for detection of a Salmonella species in a food safety application. Salmonella were inoculated into a sponge pack typically used in carcass and environmental testing, consisting of a sampling sponge moistened with Neutralizing Buffer. Three sponge pack samples were used. 30 ml Buffered Peptone Water containing 0.002% Novobiocin (BPW+N) was added to each of the three sponge pack bags, and 100 Salmonella organisms were inoculated into each bag. The cultures were then incubated at 35° C.

One (1) ml samples were taken at 30 minute intervals and centrifuged at 2100×g for 3 minutes. 900 μl supernatant was discarded and the pellet was resuspended and boiled five (5) minutes. After the presence of Neutralizing Buffer was determined to inhibit PCR, the DNA was extracted from the fluid using a suitable extraction method, such as a QIAGEN® kit. Amplification and detection was carried out by real-time PCR and detection of a fluorescence resonance energy transfer was achieved within a Roche Lightcycler. The detection was carried out utilizing PCR primers (SEQ ID NO: 1, SEQ ID NO: 2) and hybridization probes (SEQ ID NO: 3, SEQ ID NO: 4). Salmonella at a range of <10 cfu/ml was detectable with this method. Detection was determined after six (6) hours of incubation.

### EXAMPLE 5

### Detection of Listeria monocytogenes Species

The present example further exhibits the detection of Listeria monocytogenes (L. mono) utilizing PCR primers (SEQ ID NO: 9, SEQ ID NO: 10). Approximately 2-3 colonies of L. mono were transferred to distilled water, boiled five (5) minutes, and the DNA was extracted utilizing the QIAGEN® kit. Three (3) ul of a 10⁻² dilution of the extract was then transferred to capillary tubes for assay within a Roche Lightcycler. A standard optimization scheme was employed testing a range of primer concentrations (0.3 uM-0.5 uM) and MgCl₂ (2-5 mM). Amplification and detection was carried out by real-time PCR and detection of a fluorescence was performed using SYBR GREEN® dye. Detection of a PCR product (SEQ ID NO: 15) utilizing PCR primers (SEQ ID NO: 9, SEQ ID NO: 10) was seen and verified over the range of primer and MgCl₂ concentrations.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

1. A method for detecting a food-borne pathogenic bacteria located within a food sample, the method comprising the steps of:
providing a food sample containing a target DNA sequence;
providing a real-time polymerase chain reaction (PCR) primer containing a pair of nucleotide sequences;
amplifying said target DNA sequence with said PCR primer;
detecting said target DNA sequence utilizing a hybridization probe, said hybridization probe containing at least one nucleotide sequence, said nucleotide sequence located in said hybridization probe being compatible with said PCR primer nucleotide sequence;
detecting the presence of said food-borne bacteria located within said food sample, said detection carried out by amplifying said PCR primer and said hybridization probe with a nucleotide sequence compatible with said PCR primer nucleotide sequences and said hybridization probe nucleotide sequence, said detection being obtainable within 12 hours of commencement of the method.

2. The method according to claim 1 wherein said food-borne pathogenic bacteria is Salmonella sp.

3. The method according to claim 2 wherein said nucleotide sequences of said PCR primer are selected from the group consisting of:
SEQ ID NO: 1, SEQ ID NO: 2, and the complements of SEQ ID NO: 1 and SEQ ID NO: 2.

4. The method according to claim 3 wherein said nucleotide sequence of said hybridization probe is selected form the group consisting of:
SEQ ID NO: 3, SEQ ID NO: 4 and the complements of SEQ ID NO: 3 and SEQ ID NO:

5. The method according to claim 4 wherein said amplification nucleotide sequence is selected from the group consisting of:
SEQ ID NO: 13 and the complements of SEQ ID NO: 13.

6. The method according to claim 1 wherein said food-borne pathogenic bacteria is Listeria monocytogenes.

7. The method according to claim 6 wherein said nucleotide sequence of said PCR primer is selected from the group consisting of:
SEQ ID NO: 9, SEQ ID NO: 10, and the complements of SEQ ID NO: 9 and SEQ ID NO: 10.

8. The method according to claim 7 wherein said nucleotide sequence of said hybridization probe is selected form the group consisting of:
SEQ ID NO: 11, SEQ ID NO: 12 and the complements of SEQ ID NO: 11 and SEQ ID NO: 12.

9. The method according to claim 8 wherein said amplification nucleotide sequence is selected from the group consisting of:
SEQ ID NO: 15 and the complements of SEQ ID NO: 15.

10. The method according to claim 1 wherein said step of detecting said amplified target DNA is performed by fluorescence resonance energy transfer (FRET).

11. A method for detecting a food-borne pathogenic bacteria located within a food sample, the method comprising the steps of:
providing a food sample containing a target DNA sequence;
providing a real-time polymerase chain reaction (PCR) primer containing a pair of nucleotide sequences;
amplifying said target DNA sequence with said PCR primer;
detecting said target DNA sequence utilizing a hybridization probe, said hybridization probe containing at least one nucleotide sequence, said nucleotide sequence located in said hybridization probe being compatible with said PCR primer nucleotide sequences;
detecting the presence of said food-borne bacteria located within said food sample, said detection carried out by amplifying said PCR primer and said hybridization probe with a nucleotide sequence compatible with said PCR primer nucleotide sequences and said hybridization probe nucleotide sequence, said detection being performed within 6 hours of commencement of the method.

12. The method according to claim 11 wherein said food-borne pathogenic bacteria is Salmonella sp.

13. The method according to claim 12 wherein said nucleotide sequences of said PCR primer are selected from the group consisting of:
SEQ ID NO: 1, SEQ ID NO: 2, and the complements of SEQ ID NO: 1 and SEQ ID NO: 2.

14. The method according to claim 13 wherein said nucleotide sequence of said hybridization probe is selected form the group consisting of:
SEQ ID NO: 3, SEQ ID NO: 4 and the complements of SEQ ID NO: 3 and SEQ ID NO: 4.

15. The method according to claim 14 wherein said amplification nucleotide sequence is selected from the group consisting of:
SEQ ID NO: 13 and the complements of SEQ ID NO: 13.

16. The method according to claim 11 wherein said food-borne pathogenic bacteria is Listeria monocytogenes.

17. The method according to claim 16 wherein said nucleotide sequence of said PCR primer is selected from the group consisting of:
SEQ ID NO: 9, SEQ ID NO: 10, and the complements of SEQ ID NO: 9 and SEQ ID NO: 10.

18. The method according to claim 17 wherein said nucleotide sequence of said hybridization probe is selected form the group consisting of:
SEQ ID NO: 11, SEQ ID NO: 12 and the complements of SEQ ID NO: 11 and SEQ ID NO: 12.

19. The method according to claim 18 wherein said amplification nucleotide sequence is selected from the group consisting of:
SEQ ID NO: 15 and the complements of SEQ ID NO: 15.

20. The method according to claim 11 wherein said step of detecting said amplified target DNA is performed by fluorescence resonance energy transfer (FRET).
